(19) United States
(12) Patent Application Publication
(10) Pub. No.: US 2023/0176028 A1
(43) Pub. Date: Jun. 8, 2023

(54) PORTABLE MATERIALS ANALYZER

(71) Applicant: Sortera Alloys, Inc., Fort Wayne, IN (US)

(72) Inventors: Nalin Kumar, Fort Wayne, IN (US); Manuel Gerardo Garcia, JR., Fort Wayne, IN (US); Soumendu Sekhar Bhattacharjee, Mississauga (CA)

(73) Assignee: Sortera Alloys, Inc., Fort Wayne, IN (US)

(21) Appl. No.: 18/074,110

(22) Filed: Dec. 2, 2022

Related U.S. Application Data
(60) Provisional application No. 63/285,964, filed on Dec. 3, 2021.

(51) Int. Cl. G01N 33/2028

(52) U.S. Cl. CPC G01N 33/2028

(57) ABSTRACT

A portable materials analyzer is implemented in a small form factor. A sample of material pieces are retrieved from a larger container. The sample of material pieces are then analyzed by the analyzer using x-ray fluorescence, laser induced breakdown spectroscopy, a vision system with artificial intelligence, etc. The number of the material pieces within the sample is a substantially small percentage of a total number of the material scrap pieces within the container. The analyzer then determines whether the classification of the material pieces within the sample corresponds to an expected classification of the entirety of material pieces within the container.

This application claims priority to U.S. provisional patent application Ser. No. 63/285,964, which is hereby incorporated by reference herein.

### TECHNICAL FIELD

This invention relates to analyzing the composition of materials, and more specifically, analyzing the composition of materials using a small sample of such materials.

### BACKGROUND

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

Recycling is the process of collecting and processing materials that would otherwise be thrown away as trash, and turning them into new products. Recycling has benefits for communities and for the environment, since it reduces the amount of waste sent to landfills and incinerators, conserves natural resources, increases economic security by tapping a domestic source of materials, prevents pollution by reducing the need to collect new raw materials, and saves energy.

Scrap metals are often shredded, and thus require sorting to facilitate reuse of the metals. By sorting the scrap metals, metal is reused that may otherwise go to a landfill. Additionally, use of sorted scrap metal leads to reduced pollution and emissions in comparison to refining virgin feedstock from ore. Scrap metals may be used in place of virgin feedstock by manufacturers if the quality of the sorted metal meets certain standards. The scrap metals may include types of ferrous and nonferrous metals, heavy metals, high value metals such as nickel or titanium, cast or wrought metals, and other various alloys.

The recycling of aluminum (Al) scrap is a very attractive proposition in that up to 95% of the energy costs associated with manufacturing can be saved when compared with the laborious extraction of the more costly primary aluminum. Primary aluminum is defined as aluminum originating from aluminum-enriched ore, such as bauxite. At the same time, the demand for aluminum is steadily increasing in markets, such as car manufacturing, because of its lightweight properties. As a result, there are certain economies available to the aluminum industry by developing a well-planned yet simple recycling plan or system. The use of recycled material would be a less expensive metal resource than a primary source of aluminum. As the amount of aluminum sold to the automotive industry (and other industries) increases, it will become increasingly necessary to use recycled aluminum to supplement the availability of primary aluminum.

Correspondingly, it is particularly desirable to efficiently separate aluminum scrap metals into alloy families, since mixed aluminum scrap of the same alloy family is worth much more than that of indiscriminately mixed alloys. For example, in the blending methods used to recycle aluminum, any quantity of scrap composed of similar, or the same, alloys and of consistent quality, has more value than scrap consisting of mixed aluminum alloys. Within such aluminum alloys, aluminum will always be the bulk of the material. However, constituents such as copper, magnesium, silicon, iron, chromium, zinc, manganese, and other alloy elements provide a range of properties to alloyed aluminum and provide a means to distinguish one aluminum alloy from the other. Each individual aluminum alloy is a mixture of alloys in which aluminum is the predominant metal. Various other alloys, including Magnesium (Mg), Copper (Cu), Silicon (Si), Zinc (Zn), and other metals, are used to create each distinct aluminum alloy. As a result, each individual aluminum alloy has its own distinct chemistry and mechanical properties (and ranges) such as, tensile strength, yield strength, elongation, and other physical properties.

The Aluminum Association is the authority that defines the allowable limits for aluminum alloy chemical composition. The data for the aluminum wrought alloy chemical compositions is published by the Aluminum Association in “International Alloy Designations and Chemical Composition Limits for Wrought Aluminum and Wrought Aluminum Alloys,” which was updated in January 2015, and which is incorporated by reference herein. In general, according to the Aluminum Association, the 1xxx series of wrought aluminum alloys is composed essentially of pure aluminum with a minimum 99% aluminum content by weight; the 2xxx series is wrought aluminum principally alloyed with copper (Cu); the 3xxx series is wrought aluminum principally alloyed with manganese (Mn); the 4xxx series is wrought aluminum alloyed with silicon (Si); the 5xxx series is wrought aluminum primarily alloyed with magnesium (Mg); the 6xxx series is wrought aluminum principally alloyed with magnesium and silicon; the 7xxx series is wrought aluminum primarily alloyed with zinc (Zn); and the 8xxx series is a miscellaneous category.

The Aluminum Association also has a similar document for cast aluminum alloys. The 1xx series of cast aluminum alloys is composed essentially of pure aluminum with a minimum 99% aluminum content by weight; the 2xx series is cast aluminum principally alloyed with copper; the 3xx series is cast aluminum principally alloyed with silicon plus copper and/or magnesium; the 4xx series is cast aluminum principally alloyed with silicon; the 5xx series is cast aluminum principally alloyed with magnesium; the 6xx series is an unused series; the 7xx series is cast aluminum principally alloyed with zinc; the 8xx series is cast aluminum principally alloyed with tin; and the 9xx series is cast aluminum alloyed with other elements. Examples of cast alloys utilized for automotive parts include 380, 384, 356, 360, and 319. For example, recycled cast alloys 380 and 384 can be used to manufacture vehicle engine blocks, transmission cases, etc. Recycled cast alloy 356 can be used to manufacture aluminum alloy wheels. And, recycled cast alloy 319 can be used to manufacture transmission blocks.

### BRIEF DESCRIPTION OF THE DRAWINGS

FIG. 1 illustrates a simplified schematic diagram of a system configured in accordance with embodiments of the present disclosure.

FIG. 2 illustrates a flowchart diagram configured in accordance with embodiments of the present disclosure.

FIG. 3 illustrates a block diagram of a data processing system configured in accordance with embodiments of the present disclosure.

FIG. 4 illustrates a block diagram of analyzer configured in accordance with embodiments of the present disclosure.

FIG. 5 illustrates a cutaway side view of an exemplary analyzer configured in accordance with embodiments of the present disclosure.

### DETAILED DESCRIPTION

Various detailed embodiments of the present disclosure are disclosed herein. However, it is to be understood that the disclosed embodiments are merely exemplary of the disclosure, which may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to employ various embodiments of the present disclosure.

As used herein, a “material” may include a chemical element, a compound or mixture of chemical elements, wherein the complexity of a compound or mixture may range from being simple to complex. As used herein, “element” means a chemical element of the periodic table of elements, including elements that may be discovered after the filing date of this application. As used herein, “materials” may include any object, including but not limited to, airbag modules, metals (ferrous and nonferrous), metal alloys, novel alloys, super alloys (e.g., nickel super alloys), plastics (including, but not limited to PCB, HDPE, UHMWPE, and various colored plastics), rubber, foam, glass (including, but not limited to borosilicate or soda lime glass, and various colored glass), ceramics, paper, cardboard, Teflon, PE, bundled wires, insulation covered wires, rare earth elements, leaves, wood, plants, parts of plants, textiles, bio-waste, packaging, electronic waste (“e-waste”) such as electronic equipment and PCB boards, batteries and accumulators, shredded material pieces of end-of-life vehicles, pre-consumer scrap (“Clip”), mining, construction, and demolition waste, crop wastes, forest residues, purpose-grown grasses, woody energy crops, microalgae, food waste, hazardous chemical and biomedical wastes, construction debris, farm wastes, biogenic items, non-biogenic items, objects with a carbon content, organic materials (e.g., food, fluids, oils, carbohydrates, fats, proteins, animal waste, human waste, etc.), high-end composite materials (e.g., fiberglass, low-weight carbon fiber composites), agriculture materials (e.g., yard trimmings, leaves, dirt, soil, rocks, etc.), any other objects that may be found within municipal solid waste, and any other objects, items, or materials disclosed herein, including further types or classes of any of the foregoing that can be distinguished from each other by one or more sensors, including but not limited to, any of the sensor technologies disclosed herein. Within this disclosure, the terms “scrap,” “scrap pieces,” “materials,” “material pieces,” and “pieces” may be used interchangeably.

As used herein, the terms “identify,” “classify,” and “analyze,” and the terms “identification,” “classification,” and “analysis,” and any derivatives of the foregoing, may be used interchangeably. As used herein, to “analyze” a material piece is to determine the chemical composition of the material piece. For example, in accordance with certain embodiments of the present disclosure, a sensor system (as further described herein) may be configured to collect, or capture, as the case may be, any type of information (e.g., characteristics) for analyzing materials, including but not limited to, color, texture, hue, shape, brightness, weight, density, composition, size, uniformity, manufacturing type, chemical signature, radioactive signature, transmissivity to light, sound, or other signals, and reaction to stimuli such as various fields, including emitted and/or reflected electromagnetic radiation (“EM”) of the material pieces.

The types or classes (i.e., classification) of materials may be user-definable and not limited to any known classification of materials. The granularity of the types or classes may range from very coarse to very fine. For example, the types or classes may include plastics, ceramics, glasses, metals, and other materials, where the granularity of such types or classes is relatively coarse; different metals and metal alloys such as, for example, zinc, copper, brass, chrome plate, and aluminum, where the granularity of such types or classes is finer; or between specific types of metal alloys, where the granularity of such types or classes is relatively fine.

It should be noted that the materials to be analyzed may have irregular sizes and shapes. For example, such materials may have been previously run through some sort of shredding mechanism that chops up the materials into such irregularly shaped and sized pieces (producing scrap pieces). In accordance with embodiments of the present disclosure, the material pieces may include scrap pieces of end-of-life vehicles, which have been passed through some sort of shredding mechanism. Typically, the scrap pieces are of a size no more than a few inches in diameter in any direction.

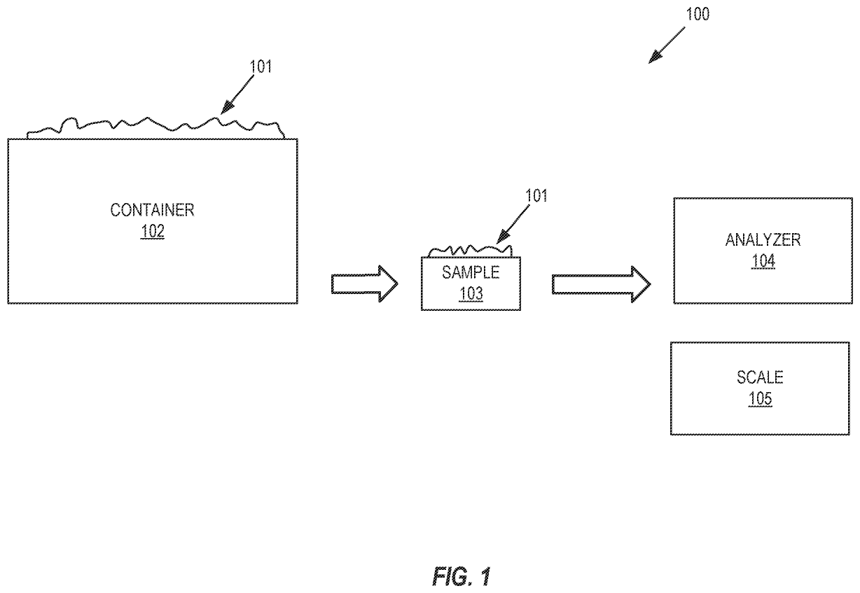

FIG. 1 illustrates a system 100 configured in accordance with embodiments of the present disclosure that provides for the analysis of a sample of material pieces retrieved from a container. For example, a container 102 of some sort may contain one or more different types or classes of material pieces 101. The material pieces 101 may be any of the materials disclosed herein, or any other types or classes of materials that could be contemplated for analysis using the system 100. The container 102 may be any type of receptacle holding such material pieces 101 (e.g., a large commercial shipping container (for example, an ISO shipping container, such as a 20-foot shipping container having internal measurements of 19 ft. 4 in. long; 7 ft. 8 in. wide; 7 ft. 10 in. high), a truckload of such material pieces 101, a railway car, etc.). Nevertheless, the container 102 is of a sufficiently large size that it is impractical to any reasonable degree to analyze every material piece within the container 102 (assuming the container is substantially filled with the material pieces).

The business entity selling, delivering, and/or shipping the container 102 of material pieces 101 and/or the business entity receiving and/or purchasing the container 102 of material pieces 101 may desire to have some sort of assurances that at least substantially all of the material pieces 101 within the container 102 have a certain or specific chemical composition(s) (e.g., as specified within the purchasing/shipping manifest or documents). For example, the material pieces 101 may be a plurality of scrap pieces of one or more types of aluminum alloy, which may be specified (e.g., within the purchasing/shipping manifest or documents) to have a specific chemical composition(s) (e.g., a specific aluminum alloy such as described herein).

Since it may be impractical to analyze every piece of the material pieces 101 (e.g., the container 102 is very large and may contain several tons of the material pieces 101), the system 100 is configured so that a sample 103 of the material pieces 101 is retrieved from the container 102 for analysis by the analyzer 104. For example, the sample 103 may be a relatively small portion (e.g., several pounds) of the material pieces 101 (i.e., the number of material pieces within the sample 103 is a substantially small percentage of a total number of the material scrap pieces within the container 102). In accordance with some embodiments of the present disclosure, the sample 103 contains less than 50%, less than 40%, less than 30%, less than 20%, less than 10%, less than 5%, or less than 1% of the total amount or number of the material pieces within the container 102. Since it may be assumed that such a sample 103 of the material pieces 101 should represent an entirety of the material pieces 101 within the entire container 102, an analysis of the sample 103 of the material pieces 101 may be used (e.g., relied upon) for representing the chemical composition(s) of all of the material pieces 101 within the container 102. The material pieces 101 within the sample 103 are then analyzed by the analyzer 104, which may be configured to output the chemical composition(s) associated with the material pieces 101 within the sample 103.

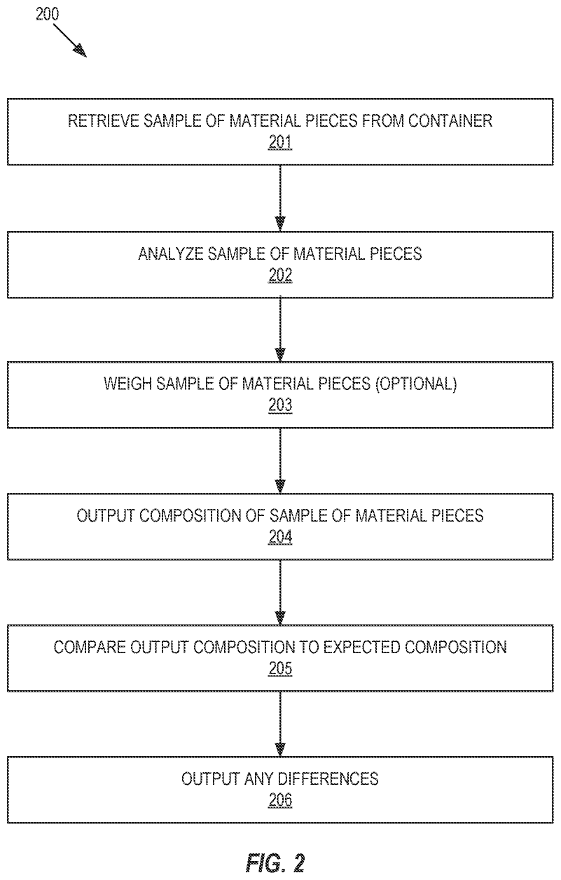

Referring next to FIG. 2, there is illustrated a flowchart diagram of a process 200 configured in accordance with embodiments of the present disclosure for use within the system 100. In the process block 201, a sample 103 of the material pieces 101 is retrieved (e.g., manually) from the container 102. In the process block 202, the sample 103 of the material pieces 101 is analyzed by the analyzer 104 as further described herein. In the optional process block 203, the sample 103 of the material pieces 101 may be weighed by an appropriate scale 105. In the process block 204, the analyzer may be configured to determine and output compositions of the material pieces 101 contained within the sample 103. Such compositions may be of each of the material pieces 101 within the sample 103 and/or an aggregate composition of all of the material pieces 101 within the sample 103.

An aggregate composition may be performed since the aggregate chemical composition of all or at least some of the material pieces 101 in the sample 103 is now known, and the total weight of the material pieces 101 in the sample 103 is also known if the weight is measured in the process block 203.

In the process block 205, the determined composition(s) of all (or at least some) or each of the material pieces 101 in the sample 103 may be compared to an expected composition(s) of the material pieces 101 within the container 102 (for example, as specified within the purchasing/shipping documents). In the process block 206, the result of this comparison may be output, which may include a side-by-side comparison of the percentages of each of the elements, and which may also include highlighting any differences that are greater than a predetermined specified threshold (which could be set to highlight that the container 102 may not contain material pieces 101 with the chemical composition(s) specified within the purchasing/shipping documents).

In accordance with embodiments of the present disclosure, the analyzer 104 may be any type of device or apparatus that is capable of analyzing and determining the chemical compositions of the material pieces 101 contained within the sample 103.

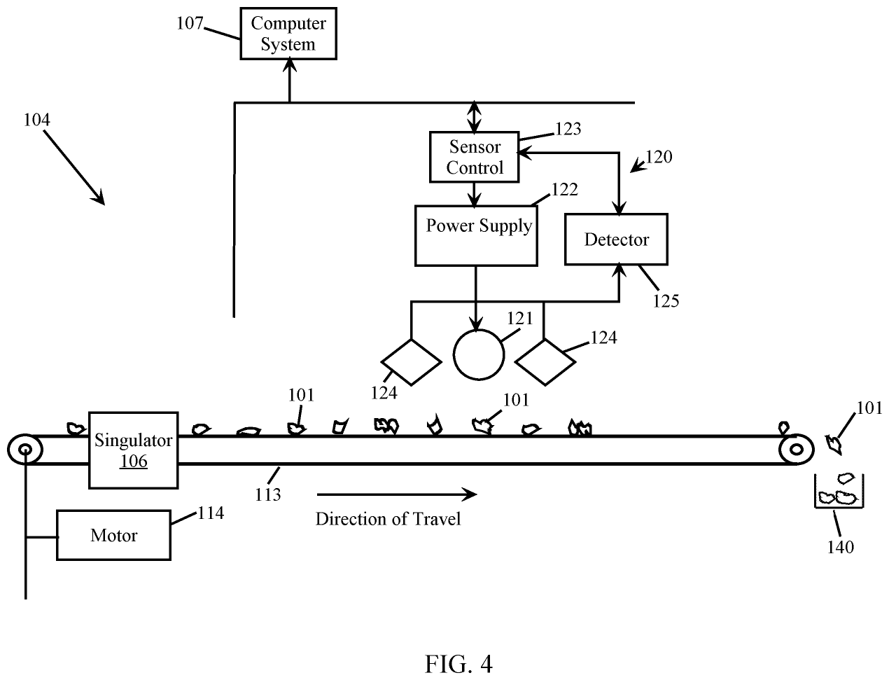

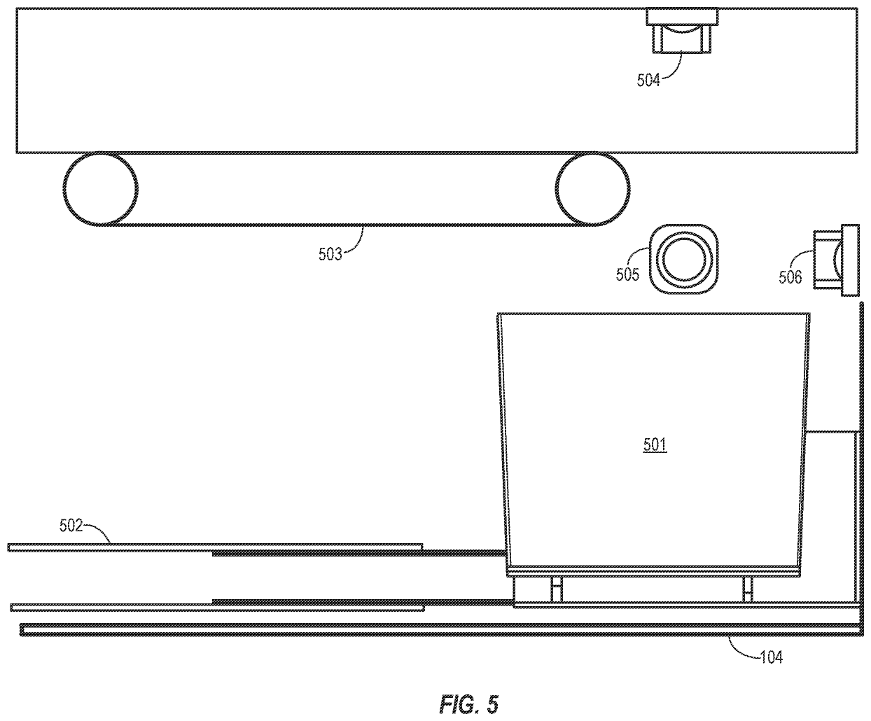

FIG. 4 illustrates a non-limiting example of components that may be implemented within an analyzer 104, which may be configured in accordance with various embodiments of the present disclosure. The components described with respect to FIG. 4 may be packaged into a configuration that is relatively small and may be portable (e.g., so that it could rest on a conventional table or workbench, such as described with respect to FIG. 5).

A conveyor belt 113 may be implemented to convey individual material pieces 101 through the analyzer 104 after they have been deposited onto the conveyor belt 113 so that each of the individual material pieces 101 can be analyzed. The conveyor belt 113 may be a conventional endless belt conveyor employing a conventional belt motor 114. In accordance with alternative embodiments of the present disclosure, some sort of suitable feeder mechanism may be used to feed the material pieces 101 onto the conveyor belt 113. In accordance with alternative embodiments of the present disclosure, the material pieces may be positioned into at least one singulated (i.e., single file) stream, which may be performed by an active or passive singulator 106. An example of a passive singulator is further described in U.S. Pat. No. 10,207,296.

In accordance with embodiments of the present disclosure, the analyzer 104 is configured with one or more analyzer devices used to analyze the material pieces 101. An analyzer device may be configured with any type of sensor technology, including analyzer devices utilizing irradiated or reflected electromagnetic radiation (e.g., utilizing the visual spectrum, infrared (“IR”), Fourier Transform IR (“FTIR”), Forward-looking Infrared (“FLIR”), Very Near Infrared (“VNIR”), Near Infrared (“NIR”), Short Wavelength Infrared (“SWIR”), Long Wavelength Infrared (“LWIR”), Medium Wavelength Infrared (“MWIR”), X-Ray Transmission (“XRT”), Gamma Ray, Ultraviolet, X-Ray Fluorescence (“XRF”), Laser Induced Breakdown Spectroscopy (“LIBS”), laser ablation using a laser used for tattoo removal, Raman Spectroscopy, Anti-stokes Raman Spectroscopy, Gamma Spectroscopy, Hyperspectral Spectroscopy (e.g., any range beyond visible wavelengths), Acoustic Spectroscopy, NMR Spectroscopy, Microwave Spectroscopy, Terahertz Spectroscopy, including one-dimensional, two-dimensional, or three-dimensional imaging with any of the foregoing), or by any other type of technology, including but not limited to, chemical or radioactive. Implementation of an XRF system is further described in U.S. Pat. No. 10,207,296, which is hereby incorporated by reference herein. Nevertheless, any of the sensor technologies disclosed herein may be implemented in one or more analyzer devices within the analyzer 104 to collect or capture information (e.g., characteristics) particularly associated with each of the material pieces, whereby that captured information may then be used to analyze the material pieces.

It should be noted that embodiments of the present disclosure may be implemented with any combination of analyzer devices utilizing any one or more of the sensor technologies disclosed herein, or any other sensor technologies currently available or developed in the future. Furthermore, embodiments of the present disclosure may include any combinations of one or more analyzer devices in which the outputs of such analyzer devices are used by an artificial intelligence system (as further disclosed herein) in order to analyze the material pieces 101.

With respect to certain sensor technologies, the one or more analyzer devices 120 may include an energy emitting source 121, which may be powered by a power supply 122, for example, in order to stimulate a response from each of the material pieces 101. As each material piece 101 passes within proximity to the emitting source 121, the analyzer device 120 may emit an appropriate sensing signal towards the material piece 101. One or more detectors 124 may be positioned and configured to sense/detect one or more physical characteristics from the material piece 101 in a form appropriate for the type of utilized sensor technology. The one or more detectors 124 and the associated detector electronics 125 capture the received sensed characteristics to perform signal processing thereon and produce digitized information representing the sensed characteristics, which are then analyzed in accordance with certain embodiments of the present disclosure, and which may be used in order to analyze each of the material pieces 101.

The analyzer 104 may also include a receptacle or bin 140 that receives the material pieces 101 after the analysis. A scale 105 may be associated with the receptacle/bin 140 to weigh the material pieces 101.

The emitting source 121 may be located above the detection area (i.e., above the conveyor belt 113); however, certain embodiments of the present disclosure may locate the emitting source 121 and/or detectors 124 in other positions that still produce acceptable sensed/detected physical characteristics. Note that the one or more analyzer devices 120 may be positioned at the end of the conveyor belt 113 to analyze the material pieces 101 after they have fallen from the edge of the conveyor belt 113 (for example, see FIG. 5).

In accordance with certain embodiments of the present disclosure, an analyzer device may utilize a vision system to analyze each of the material pieces 101. The vision system 11 may be configured to perform certain types of identification (e.g., analysis) of all or a portion of the material pieces 101. For example, such a vision system may be utilized to collect or capture information about each of the material pieces 101. For example, the vision system may be configured (e.g., with an artificial intelligence (“AI”) system) to collect or capture any type of information that can be utilized within the analyzer 104 to classify the material pieces 101 as a function of a set of one or more (user-defined) physical characteristics, including, but not limited to, color, hue, size, shape, texture, overall physical appearance, uniformity, chemical composition, and/or manufacturing type of the material pieces 101. The vision system may capture images of each of the material pieces 101 (including one-dimensional, two-dimensional, three-dimensional, or holographic imaging), for example, by using one or more optical sensors as utilized in typical digital cameras and video equipment. Such images captured by the optical sensor(s) may then be stored in a memory device as image data. In accordance with embodiments of the present disclosure, such image data may represent images captured within optical wavelengths of light (i.e., the wavelengths of light that are observable by the typical human eye). However, alternative embodiments of the present disclosure may utilize sensors that are capable of capturing an image of a material made up of wavelengths of light outside of the visual wavelengths of the typical human eye (e.g., infrared or ultraviolet).

FIG. 5 illustrates a cutaway side view of an exemplary analyzer 104 configured in accordance with embodiments of the present disclosure. Such an analyzer 104 may be implemented within a small form factor of a metal box in which the components are mounted and located with respect to each other. Note that any one or more of the components disclosed with respect to FIG. 4 may be implemented within the exemplary analyzer 104 of FIG. 5.

The material pieces 101 may be deposited onto the conveyor belt 503 so that each of the material pieces 101 passes by one or more analyzer devices (e.g., any one or more of the analyzer devices utilizing any of the sensor technologies disclosed herein). In this exemplary embodiment, the material pieces 101 may fall off of the end of the conveyor belt 503 and into a receptacle 501 by which each of the material pieces 101 has characteristics captured by one or more analyzer devices 504 . . . 506 mounted to portions of (e.g., the internal sides) of the analyzer 104 as the material pieces 101 are in free fall from the conveyor belt 503 to the receptacle 501. When the material pieces 101 are each deposited into the receptacle 501, a scale may be implemented on which the receptacle 501 sits in order to weigh each other material pieces 101. After the process of analyzing the material pieces 101 is completed, they may be retrieved from the analyzer 104 by sliding the receptacle 501 along the track 502 to an outside of the box containing the analyzer 104. The track 502 may be similar to that used within a metal filing cabinet for allowing file drawers to be opened and closed.

Regardless of the type(s) of sensed characteristics/information captured of the material pieces, the information may then be sent to a computer system (e.g., computer system 107) to be processed (e.g., by an artificial intelligence system) in order to analyze each of the material pieces. An artificial intelligence system may implement any well-known machine learning technique or technology, including one that implements a neural network (e.g., artificial neural network, deep neural network, convolutional neural network, recurrent neural network, autoencoders, reinforcement learning, etc.), supervised learning, unsupervised learning, semi-supervised learning, reinforcement learning, self-learning, feature learning, sparse dictionary learning, anomaly detection, robot learning, association rule learning, fuzzy logic, artificial intelligence (“AI”), deep learning algorithms, deep structured learning hierarchical learning algorithms, support vector machine (“SVM”) (e.g., linear SVM, nonlinear SVM, SVM regression, etc.), decision tree learning (e.g., classification and regression tree (“CART”), ensemble methods (e.g., ensemble learning, Random Forests, Bagging and Pasting, Patches and Subspaces, Boosting, Stacking, etc.), dimensionality reduction (e.g., Projection, Manifold Learning, Principal Components Analysis, etc.) and/or deep machine learning algorithms, such as those described in and publicly available at the deeplearning.net website (including all software, publications, and hyperlinks to available software referenced within this website), which is hereby incorporated by reference herein. Non-limiting examples of publicly available machine learning software and libraries that could be utilized within embodiments of the present disclosure include Python, OpenCV, Inception, Theano, Torch, PyTorch, Pylearn2, Numpy, Blocks, TensorFlow, MXNet, Caffe, Lasagne, Keras, Chainer, Matlab Deep Learning, CNTK, MatConvNet (a MATLAB toolbox implementing convolutional neural networks for computer vision applications), DeepLearnToolbox (a Matlab toolbox for Deep Learning (from Rasmus Berg Palm)), BigDL, Cuda-Convnet (a fast C++/CUDA implementation of convolutional (or more generally, feed-forward) neural networks), Deep Belief Networks, RNNLM, RNNLIB-RNNLIB, matrbm, deeplearning4j, Eblearn.lsh, deepmat, MShadow, Matplotlib, SciPy, CXXNET, Nengo-Nengo, Eblearn, cudamat, Gnumpy, 3-way factored RBM and mcRBM, mPoT (Python code using CUDAMat and Gnumpy to train models of natural images), ConvNet, Elektronn, OpenNN, NeuralDesigner, Theano Generalized Hebbian Learning, Apache Singa, Lightnet, and SimpleDNN.

It should be understood that the present disclosure is not exclusively limited to artificial intelligence techniques. Other common techniques for material classification/identification may also be used. For instance, an analyzer device may utilize optical spectrometric techniques using multi- or hyper-spectral cameras to provide a signal that may indicate the presence or absence of a type of material by examining the spectral emissions of the material. Photographs of a material piece may also be used in a template-matching algorithm, wherein a database of images is compared against an acquired image to find the presence or absence of certain types of materials from that database. A histogram of the captured image may also be compared against a database of histograms. Similarly, a bag of words model may be used with a feature extraction technique, such as scale-invariant feature transform (“SIFT”), to compare extracted features between a captured image and those in a database. In accordance with certain embodiments of the present disclosure, instead of utilizing a training stage whereby control samples of material pieces are utilized, training of the artificial intelligence system may be performed utilizing a labeling/annotation technique (or any other supervised learning technique) whereby as data/information of material pieces are captured by an analyzer device, a user inputs a label or annotation that identifies each material piece, which is then used to create the library for use by the artificial intelligence system when analyzing material pieces. In other words, a previously generated knowledge base of characteristics captured from one or more samples of a class of materials may be accomplished by any of the techniques disclosed herein, whereby such a knowledge base is then used to automatically analyze materials.

In accordance with certain embodiments of the present disclosure, any sensed characteristics captured by any of the analyzer devices 120 disclosed herein may be input into an artificial intelligence system in order to analyze materials. For example, in an artificial intelligence system implementing supervised learning, analyzer device outputs that uniquely characterize a specific type or composition of material (e.g., a specific metal alloy) may be used to train the artificial intelligence system.

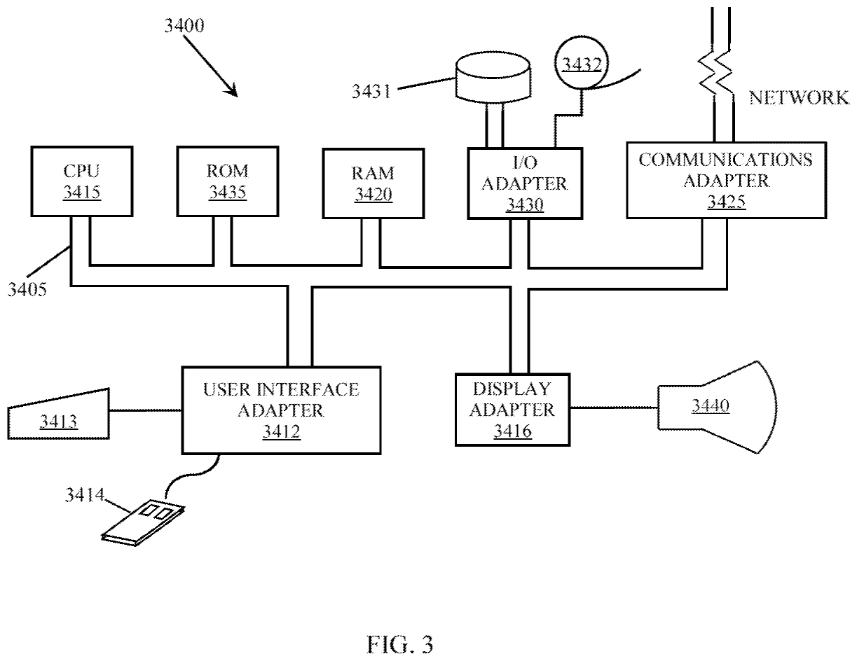

With reference now to FIG. 3, a block diagram illustrating a data processing (“computer”) system 3400 is depicted in which aspects of embodiments of the disclosure may be implemented. (The terms “computer,” “system,” “computer system,” and “data processing system” may be used interchangeably herein.) The analyzer 104 may be configured with the computer system 3400 (e.g., as the computer system 107). The computer system 3400 may employ a local bus 3405 (e.g., a peripheral component interconnect (“PCI”) local bus architecture). Any suitable bus architecture may be used such as Accelerated Graphics Port (“AGP”) and Industry Standard Architecture (“ISA”), among others. One or more processors 3415, volatile memory 3420, and non-volatile memory 3435 may be connected to the local bus 3405 (e.g., through a PCI Bridge (not shown)). An integrated memory controller and cache memory may be coupled to the one or more processors 3415. The one or more processors 3415 may include one or more central processor units and/or one or more graphics processor units and/or one or more tensor processing units. Additional connections to the local bus 3405 may be made through direct component interconnection or through add-in boards. In the depicted example, a communication (e.g., network (LAN)) adapter 3425, an I/O (e.g., small computer system interface (“SCSI”) host bus) adapter 3430, and expansion bus interface (not shown) may be connected to the local bus 3405 by direct component connection. An audio adapter (not shown), a graphics adapter (not shown), and display adapter 3416 (coupled to a display 3440 (which may be touch screen display)) may be connected to the local bus 3405 (e.g., by add-in boards inserted into expansion slots).

The user interface adapter 3412 may provide a connection for a keyboard 3413 and a mouse 3414, modem/router (not shown), and additional memory (not shown). The I/O adapter 3430 may provide a connection for a hard disk drive 3431, a tape drive 3432, and a CD-ROM drive (not shown).

One or more operating systems may be run on the one or more processors 3415 and used to coordinate and provide control of various components within the analyzer 104. The operating system(s) may be a commercially available operating system. An object-oriented programming system (e.g., Java, Python, etc.) may run in conjunction with the operating system and provide calls to the operating system from programs or programs (e.g., Java, Python, etc.) executing on the system 3400. Instructions for the operating system, the object-oriented operating system, and programs may be located on non-volatile memory 3435 storage devices, such as a hard disk drive 3431, and may be loaded into volatile memory 3420 for execution by the processor 3415.

Those of ordinary skill in the art will appreciate that the hardware in FIG. 3 may vary depending on the implementation. Other internal hardware or peripheral devices, such as flash ROM (or equivalent nonvolatile memory) or optical disk drives and the like, may be used in addition to or in place of the hardware depicted in FIG. 3. Also, any of the processes of the present disclosure may be applied to a multiprocessor computer system, or performed by a plurality of such systems 3400.

As another example, the computer system 3400 may be a stand-alone system (e.g., separate from the analyzer 104). As a further example, the computer system 3400 may be an embedded controller, which is configured with ROM and/or flash ROM providing non-volatile memory storing operating system files or user-generated data.

The depicted example in FIG. 3 and above-described examples are not meant to imply architectural limitations. Further, a computer program form of aspects of the present disclosure may reside on any computer readable storage medium (i.e., floppy disk, compact disk, hard disk, tape, ROM, RAM, etc.) used by a computer system.

As has been described herein, embodiments of the present disclosure may be implemented to perform the various functions described for analyzing material pieces. Such functionalities may be implemented within hardware and/or software, such as within one or more data processing systems (e.g., the data processing system 3400 of FIG. 3). Nevertheless, the functionalities described herein are not to be limited for implementation into any particular hardware/software platform.

As will be appreciated by one skilled in the art, aspects of the present disclosure may be embodied as a system, process, method, and/or program product. Accordingly, various aspects of the present disclosure may take the form of an entirely hardware embodiment, an entirely software embodiment (including firmware, resident software, micro-code, etc.), or embodiments combining software and hardware aspects, which may generally be referred to herein as a “circuit,” “circuitry,” “module,” or “system.” Furthermore, aspects of the present disclosure may take the form of a program product embodied in one or more computer readable storage medium(s) having computer readable program code embodied thereon. (However, any combination of one or more computer readable medium(s) may be utilized. The computer readable medium may be a computer readable signal medium or a computer readable storage medium.)

The flowchart and block diagrams in the figures illustrate architecture, functionality, and operation of possible implementations of systems, methods, processes, and program products according to various embodiments of the present disclosure. In this regard, each block in the flowcharts or block diagrams may represent a module, segment, or portion of code, which includes one or more executable program instructions for implementing the specified logical function(s). It should also be noted that, in some implementations, the functions noted in the blocks may occur out of the order noted in the figures. For example, two blocks shown in succession may, in fact, be executed substantially concurrently, or the blocks may sometimes be executed in the reverse order, depending upon the functionality involved.

Program instructions may be provided to one or more processors and/or controller(s) of a general purpose computer, special purpose computer, or other programmable data processing apparatus (e.g., controller) to produce a machine, such that the instructions, which execute via the processor(s) (e.g., CPU 3415) of the computer or other programmable data processing apparatus, create circuitry or means for implementing the functions/acts specified in the flowchart and/or block diagram block or blocks.

In the description herein, a flow-charted technique may be described in a series of sequential actions. The sequence of the actions, and the element performing the actions, may be freely changed without departing from the scope of the teachings. Actions may be added, deleted, or altered in several ways. Similarly, the actions may be re-ordered or looped. Further, although processes, methods, algorithms, or the like may be described in a sequential order, such processes, methods, algorithms, or any combination thereof may be operable to be performed in alternative orders. Further, some actions within a process, method, or algorithm may be performed simultaneously during at least a point in time (e.g., actions performed in parallel), and can also be performed in whole, in part, or any combination thereof.

Reference is made herein to “configuring” a device or a device “configured to” perform some function. It should be understood that this may include selecting predefined logic blocks and logically associating them, such that they provide particular logic functions, which includes monitoring or control functions. It may also include programming computer software-based logic of a control device, wiring discrete hardware components, or a combination of any or all of the foregoing. Such configured devises are physically designed to perform the specified function or functions.

To the extent not described herein, many details regarding specific materials, processing acts, and circuits are conventional, and may be found in textbooks and other sources within the computing, electronics, and software arts.

Computer program code, i.e., instructions, for carrying out operations for aspects of the present disclosure may be written in any combination of one or more programming languages, including an object oriented programming language such as Java, Smalltalk, Python, C++, or the like, conventional procedural programming languages, such as the “C” programming language or similar programming languages, programming languages such as MATLAB or LabVIEW, or any of the machine learning software disclosed herein. The program code may execute entirely on the user's computer system, partly on the user's computer system, as a stand-alone software package, partly on the user's computer system (e.g., the computer system utilized for sorting) and partly on a remote computer system (e.g., the computer system utilized to train the machine learning system), or entirely on the remote computer system or server. In the latter scenario, the remote computer system may be connected to the user's computer system through any type of network, including a local area network (“LAN”) or a wide area network (“WAN”), or the connection may be made to an external computer system (for example, through the Internet using an Internet Service Provider).

In the descriptions herein, numerous specific details are provided, such as examples of programming, software modules, user selections, network transactions, database queries, database structures, hardware modules, hardware circuits, hardware chips, controllers, etc., to provide a thorough understanding of embodiments of the disclosure. One skilled in the relevant art will recognize, however, that the disclosure may be practiced without one or more of the specific details, or with other methods, components, materials, and so forth. In other instances, well-known structures, materials, or operations may be not shown or described in detail to avoid obscuring aspects of the disclosure.

Reference throughout this specification to “an embodiment,” “embodiments,” or similar language means that a particular feature, structure, or characteristic described in connection with the embodiments is included in at least one embodiment of the present disclosure. Thus, appearances of the phrases “in one embodiment,” “in an embodiment,” “embodiments,” “certain embodiments,” “various embodiments,” and similar language throughout this specification may, but do not necessarily, all refer to the same embodiment. Furthermore, the described features, structures, aspects, and/or characteristics of the disclosure may be combined in any suitable manner in one or more embodiments. Correspondingly, even if features may be initially claimed as acting in certain combinations, one or more features from a claimed combination can in some cases be excised from the combination, and the claimed combination can be directed to a sub-combination or variation of a sub-combination.

Benefits, advantages, and solutions to problems have been described above with regard to specific embodiments. However, the benefits, advantages, solutions to problems, and any element(s) that may cause any benefit, advantage, or solution to occur or become more pronounced may be not to be construed as critical, required, or essential features or elements of any or all the claims. Further, no component described herein is required for the practice of the disclosure unless expressly described as essential or critical.

Herein, the term “or” may be intended to be inclusive, wherein “A or B” includes A or B and also includes both A and B. As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The terminology used herein is for the purpose of describing particular embodiments only and is not intended to be limiting of the disclosure. As used herein, the singular forms “a,” “an,” and “the” may be intended to include the plural forms as well, unless the context clearly indicates otherwise.

The corresponding structures, materials, acts, and equivalents of all means or step plus function elements in the claims below may be intended to include any structure, material, or act for performing the function in combination with other claimed elements as specifically claimed.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary.

Unless defined otherwise, all technical and scientific terms (such as acronyms used for chemical elements within the periodic table) used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

As used herein, the term “similar” refers to values that are within a particular offset or percentage of each other (e.g., 1%, 2%, 5%, 10%, etc.).

The term “coupled,” as used herein, is not intended to be limited to a direct coupling or a mechanical coupling. Unless stated otherwise, terms such as “first” and “second” are used to arbitrarily distinguish between the elements such terms describe. Thus, these terms are not necessarily intended to indicate temporal or other prioritization of such elements.

What is claimed is:

1. A method comprising:
retrieving a sample of materials from a container, wherein the sample contains substantially less than a total amount of the materials within the container;
analyzing the materials to determine chemical compositions of each of the materials within the sample; and
comparing the determined chemical compositions to an expected chemical composition, wherein the expected chemical composition is the chemical composition for all of the materials within the container.

2. The method as recited in claim 1, wherein the materials comprise Twitch.

3. The method as recited in claim 2, wherein the analyzing the materials is performed by an XRF system.

4. The method as recited in claim 1, wherein the analyzing the materials is performed by a LIBS system.

5. The method as recited in claim 1, wherein the analyzing the materials is performed by a vision system implemented with an artificial intelligence system.

6. The method as recited in claim 1, wherein a number of the materials within the sample is less than 50%, less than 40%, less than 30%, less than 20%, less than 10%, less than 5%, or less than 1% of a total number of the materials within the container.

7. The method as recited in claim 1, wherein a number of the materials within the sample is less than 5% of a total number of the materials within the container.

8. The method as recited in claim 1, wherein the analyzing the materials comprises determining a relative percentage of different elements contained within each of the materials.

9. The method as recited in claim 8, further comprising outputting whether the determination of the chemical composition of the sample of materials indicates that one or more elements have a relative percentage outside of a percentage range of the expected chemical composition.

10. The method as recited in claim 1, wherein the materials are scrap metal pieces.

11. The method as recited in claim 11, wherein the scrap metal pieces are from end-of-life vehicles.

12. The method as recited in claim 6, wherein the container is an ISO shipping container.

13. A method comprising:
retrieving a sample of material scrap pieces from a container holding the material scrap pieces, wherein a number of the material scrap pieces within the sample is a substantially small percentage of a total number of the material scrap pieces within the container;
analyzing the sample of material scrap pieces to classify the material scrap pieces within the sample; and
determining whether the classification of the material scrap pieces within the sample correspond to an expected classification of the entirety of material pieces within the container.

14. The method as recited in claim 13, wherein a number of the material scrap pieces within the sample is less than 50%, less than 40%, less than 30%, less than 20%, less than 10%, less than 5%, or less than 1% of the total number of the material scrap pieces within the container.

15. The method as recited in claim 13, wherein a number of the material scrap pieces within the sample is less than 5% of the total number of the material scrap pieces within the container.

16. The method as recited in claim 13, wherein the material scrap pieces are Twitch.

17. The method as recited in claim 13, wherein the analyzing the material scrap pieces is performed by an XRF system.

18. The method as recited in claim 13, wherein the analyzing the material scrap pieces is performed by a LIBS system.

19. The method as recited in claim 13, wherein the analyzing the material scrap pieces is performed by a vision system implemented with an artificial intelligence system.

20. The method as recited in claim 13, wherein the container is an ISO shipping container, and wherein the sample is less than 20 pounds of material scrap pieces.
